(19) United States
(12) Patent Application Publication
(10) Pub. No.: US 2021/0386877 A1
(43) Pub. Date: Dec. 16, 2021

(54) RADIOTHERAPEUTIC COMBINATION THERAPY FOR THE TREATMENT OF CANCER

(71) Applicants: Andarix Pharmaceuticals, Inc., Somerville, MA (US); Institute For Cancer Research d/b/a The Research Institute of Fox Chase Cancer Center, Philadelphia, PA (US)

(72) Inventors: Christopher P Adams, Somerville, MA (US); Martin J Edelman, Philadelphia, PA (US); Erica Golemis, Philadelphia, PA (US); Yanis Boumber, Philadelphia, PA (US)

(21) Appl. No.: 17/420,671

(22) PCT Filed: Jan. 10, 2019
(86) PCT No.: PCT/US19/13042

§ 371 Date: Jul. 3, 2021

(51) Int. Cl. A61K 51/08; A61P 35/00; A61K 31/4745; A61K 31/19

(52) U.S. Cl. CPC A61K 51/088; A61K 31/19; A61K 31/4745; A61P 35/00

(57) ABSTRACT

The present invention provides a dosage-specific treatment for lung and neuroendocrine tumors with a targeted radiotherapeutic somatostatin analogue, Re-P2045 in combination with topotecan or a histone deacetylase inhibitor. Patients are selected and dosing is determined using a technetium-labeled somatostatin analogue, ^{99m}Tc-P2045.

### CROSS REFERENCE TO RELATED APPLICATIONS

None.

### GOVERNMENT RIGHTS

None.

### FIELD OF THE INVENTION

The present invention relates to the treatment of cancer expressing somatostatin receptor subtype 2 (SSTR2). In particular, the invention provides therapeutic combinations that include P2045 chelated to a radiotherapeutic rhenium isotope and the topoisomerase inhibitor topotecan or a histone deacetylase inhibitor for the treatment of SSTR2-expressing cancers such as, for example, lung and neuroendocrine cancers in a patient, or alleviating one or more symptoms thereof. The present invention also provides kits that include the therapeutic combinations of the invention.

### BACKGROUND

While there have been significant advances in the treatment of many solid tumors, including lung cancer and neuroendocrine (NE) tumors of pulmonary and gastrointestinal (GI) tract origin, there are essentially no curative treatments and very limited palliative therapies available for patients who relapse after initial therapy or for patients who present with metastatic disease. Neuroenodocrine cancers, defined by the presence of neuroendocrine markers such as chormogranin, synaptophysin, etc. may originate from pulmonary, GI, gynecological (GYN), genitourinary (GU) and other tissues. Well-differentiated tumors may be cured surgically, however, poorly differentiated and/or metastatic disease, for which the term ‘neuroendocrine carcinoma’ is now restricted, is usually lethal. Though these diseases have diverse tissues of origin, they share a similar biology. All are individually uncommon (small cell lung cancer, the most common type accounts for approximately 25-35,000 cases/year in the United States, with <5% of patients surviving 5 years). The other diseases are much less common (each constituting <3% of all malignancies in individual anatomical sites) and the overall prevalence of metastatic NE tumors is less than 200,000 in the U.S., thereby falling under the umbrella of ‘orphan disease.’ It is highly unlikely that targeting of any single pathway or even multiple pathways is likely to be of major benefit. In addition, tumor heterogeneity, both within the primary tumor as well as between the primary tumor and metastatic lesions, is a significant problem that limits any targeted treatment.

There have been relatively few advances in systemic therapy of metastatic NE tumors in the past two decades. Topotecan approved in its intravenous (IV) formulation in 1996 and an oral formulation in 2007, and nivolumab have been the only agents approved for small cell lung carcinoma (SCLC) in the past 25 years. GI neuroendocrine tumors have been managed with a variety of agents including interferons, somatatostatin analogues, and recently sunitinib and temsirolimus. These approaches have demonstrated modest efficacy, decreasing symptomatology in hormone producing disease with some improvements in survival. Importantly, these studies were conducted in well-differentiated or moderately differentiated NE tumors. Poorly differentiated NE tumors (i.e., neuroendocrine carcinomas) have a different biology than well or moderately differentiated NE tumors and respond to treatments utilized for small cell carcinoma of the lung. They must therefore be considered a different disease entity. It is currently recommended that well differentiated and poorly differentiated neuroendocrine tumors should be studied separately.

No prospective trials have been conducted in extrapulmonary small cell carcinomas (EPSC), and the management is essentially the same as for SCLC of the lung. Large cell neuroendocrine cancer (LCNEC) of the lung is generally managed in a fashion similar to non-small cell lung cancer, but is reported to have a worse outcome.

There is therefore a need for new therapies, particularly those that have reduced side effects, have a reduced emergence of resistance, reduced treatment periods and/or enhanced cure or survival rates.

### SUMMARY OF THE INVENTION

A combination therapy approach that can both kill tumor cells which express a particular target as well as surrounding cells that may either not express that target or are resistant to inhibition of a specific target is disclosed herein. Including a radiopharmaceutical agent as part of the combination therapy uniquely meets treatment requirements in that it is a simultaneously targeted treatment as well as being capable of (depending upon the nature of the radioisotope) killing surrounding tumor cells and supporting stroma that may not be available to, or are resistant to, other chemotherapeutic drugs. As described herein, beta-emitting radiopharmaceuticals such as ¹⁸⁸Re-P2045 and ¹⁸⁶Re-P2045 have an adequate path length to kill surrounding malignant cells that do not express or are not directly bound by the agent through the “cross fire” effect.

In an aspect, a method for the treatment of a somatostatin subtype 2-expressing cancer in a patient comprises administering: (i) an effective amount of a compound having the formula I:

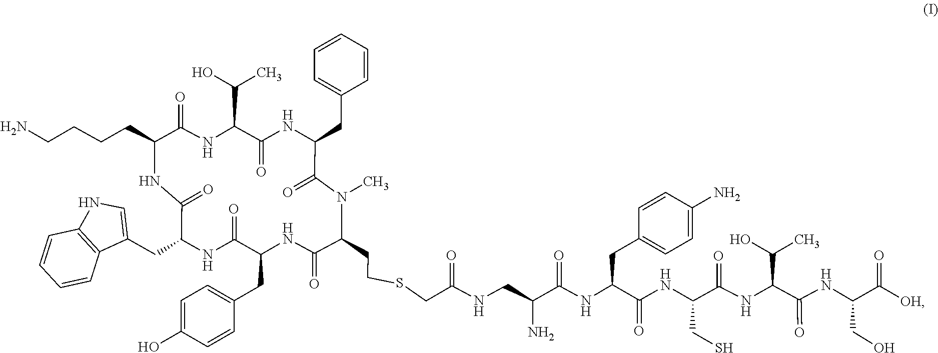 [SMILES: CC(O)[C@H](NC(=O)[C@H](CS)NC(=O)[C@H](Cc1ccc(N)cc1)NC(=O)[C@@H](N)CNC(=O)CSCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N[C@@H](Cc2ccccc2)C(=O)N1C)C(=O)N[C@@H](CO)C(=O)O]

or a pharmaceutically acceptable salt thereof, wherein the compound of formula I is bonded to a radiotherapeutic isotope of rhenium to form a rhenium chelate; and (ii) an effective amount of topotecan, or a pharmaceutically acceptable salt thereof; or an effective amount of a histone deacetylase inhibitor (HDACi), or a pharmaceutically acceptable salt thereof.

In an embodiment, the HDACi is selected from the group consisting of hydroxamic acids, short chain fatty acids, benzamides, cyclic tetrapeptides, sirtuins inhibitors and combinations thereof. In an embodiment, the HDACi is a short chain fatty acid.

In an embodiment, the HDACi is selected from the group consisting of Trichostatin A, SAHA, Belinostat, Panabiostat, Givinostat, Resminostat, Abexinostat, Quisinostat, Rocilinostat, Practinostat, CHR-3996, valproic acid, butyric acid, phenylbutyric acid, Entinostat, Tacedinaline, 4SC202, Mocetinostat, Romidepsin, nicotinamide, Sirtinol, Cambinol, EX-527 and combinations thereof. In an embodiment, the HDACi is selected from the group consisting of valproic acid, butyric acid, phenylbutyric acid and combinations thereof. In an embodiment, the HDACi is valproic acid.

In an embodiment, the rhenium chelate is a compound having the formula II:

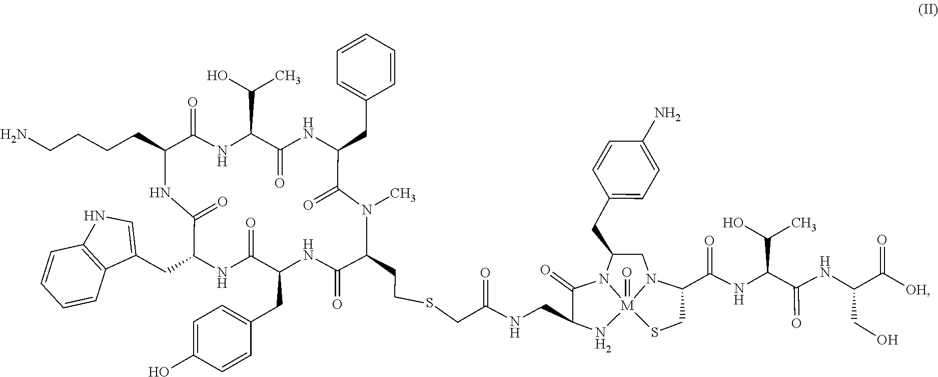

wherein M is a radiotherapeutic isotope of rhenium.

In an embodiment, the radiotherapeutic isotope of rhenium is ¹⁸⁸Re and/or ¹⁸⁶Re.

In an embodiment, the radiotherapeutic isotope of rhenium is administered at a dose of from 80 mCi/m² to 250 mCi/m². Accordingly, the dose of the radiotherapeutic isotope of rhenium can be 80 mCi/m², 90 mCi/m², 100 mCi/m², 110 mCi/m², 120 mCi/m², 130 mCi/m², 140 mCi/m², 150 mCi/m², 160 mCi/m², 170 mCi/m², 180 mCi/m², 190 mCi/m², 200 mCi/m², 210 mCi/m², 220 mCi/m², 230 mCi/m², 240 mCi/m², or 250 mCi/m².

In an embodiment, the radiotherapeutic isotope of rhenium is administered no more than once within a 28-day period.

In an embodiment, the method additionally includes administering a technetium chelate having the formula II:

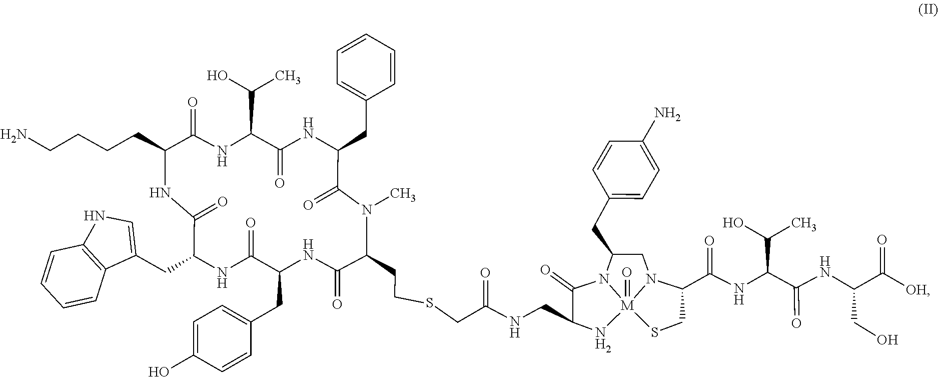

where M is ^{99m}Tc and the technetium chelate is administered before the administration of the rhenium chelate.

In an embodiment, the technetium chelate of formula II is administered no more than 2 weeks before the administration of the rhenium chelate of formula II.

In an embodiment, the method further includes administering a supplemental amino acid solution intravenously 4 hours to 24 hours prior to administering the rhenium chelate. Examples include Aminosyn®, Aminosyn®II, FreAmine®, FreAmine®II, FreAmine®III, and Travasol®. In a further embodiment, the amino acid solution comprises lysine.

In an embodiment, topotecan is administered intravenously. In an example, the effective amount of topotecan delivered intravenously is from 1 mg/m²/day to 1.5 mg/m²/day. The intravenous dosing of topotecan can be for 1 day, 2 days, 3 days, 4 days, or 5 days. In an embodiment, topotecan is administered three times within a 28-day period. In an embodiment, the rhenium chelate is administered 4 to 6 days after the administration of the first dose of topotecan.

In an embodiment, topotecan is administered orally. In an example, the effective amount of topotecan delivered orally is from 1.8 mg/m²/day to 2.3 mg/m²/day. The oral dosing of topotecan can be for 1 day, 2 days, 3 days, 4 days, or 5 days.

In an embodiment, the effective amount of histone deacetylase inhibitor is administered orally before the administration of the rhenium chelate. In an embodiment, the histone deacetylase inhibitor is administered at a daily dose of from 75 mg/kg to 150 mg/kg. In an embodiment, the daily dose of the histone deacetylase inhibitor is divided into 2 or 3 portions and is administered over 7 days.

In an embodiment, the somatostatin subtype 2-expressing cancer is selected from a small cell lung carcinoma or a neuroendocrine carcinoma, such as a low-grade neuroendocrine tumor of GI origin or a gastroenteropancreatic neuroendocrine tumor (GEP-NET) of the foregut (gastroduodenal), midgut (distal small intestine and proximal colon) or hindgut (distal colorectal and pancreas). In a further embodiment, the somatostatin subtype 2-expressing cancer is metastatic.

In an aspect, a method of treating a somatostatin subtype 2-expressing tumor in a patient comprises the steps of: a) chelating ^{99m}Tc with P2045 to form a diagnostic agent; b) administering an effective diagnostic amount of the diagnostic agent to the patient; c) detecting ^{99m}Tc accumulated in the patient, thereby determining the presence of the somatostatin-expressing tumor; d) chelating ¹⁸⁸Re and/or ¹⁸⁶Re with P2045 to form a radiotherapeutic agent; and e) administering a therapeutically effective amount of the radiotherapeutic agent to the patient, wherein the therapeutically effective amount is calculated according to the patient's whole body surface area and the dose is from 80 mCi/m² to 250 mCi/m².

In an aspect, a kit useful for making a radiotherapeutic preparation, comprises (i) a first sealed vial containing a predetermined quantity of a compound having the formula I:

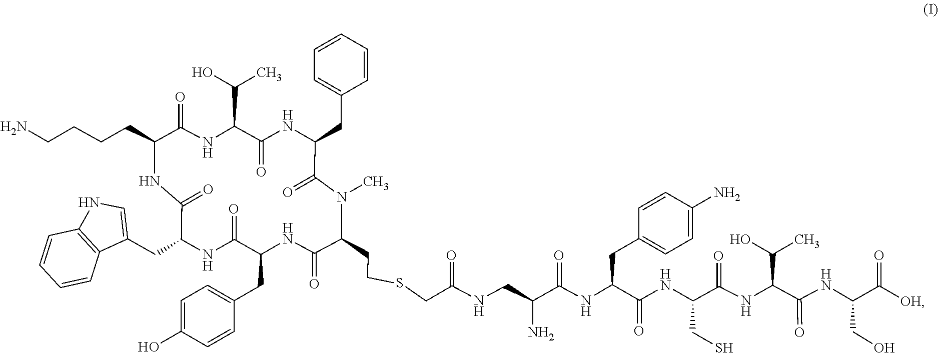 [SMILES: CC(O)[C@H](NC(=O)[C@H](CS)NC(=O)[C@H](Cc1ccc(N)cc1)NC(=O)[C@@H](N)CNC(=O)CSCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N[C@@H](Cc2ccccc2)C(=O)N1C)C(=O)N[C@@H](CO)C(=O)O]

or a pharmaceutically acceptable salt thereof, and an effective amount of a reducing agent to affect chelation of the compound of formula I with ¹⁸⁶Re and/or ¹⁸⁸Re;
(ii) a second vial containing effective stabilizing amounts of gentisic acid and ascorbic acid; and
(iii) a third vial containing a predetermined quantity of topotecan.

In an embodiment, the quantity of a compound having formula I in the first vial is from 45 μg to 60 μg of anhydrous material. In an embodiment, the quantity of a compound having formula I in the first vial is about 70 μg of P2045 trifluoroacetate.

In an embodiment, the reducing agent is SnCl₂. In an embodiment, the amount of SnCl₂ is about 850 μg.

In an embodiment, the gentisic acid in the second vial is in the form of gentisic acid, sodium salt, monohydrate. In an embodiment, the quantity of gentisic acid, sodium salt, monohydrate is about 20 mg. In an embodiment, the quantity of ascorbic acid in the second vial is about 10 mg.

In an embodiment, the predetermined amount of topotecan in the third vial is 4 mg.

In an aspect, a kit useful for making a radiotherapeutic preparation, comprises (i) a first sealed vial containing a predetermined quantity of a compound having the formula I:

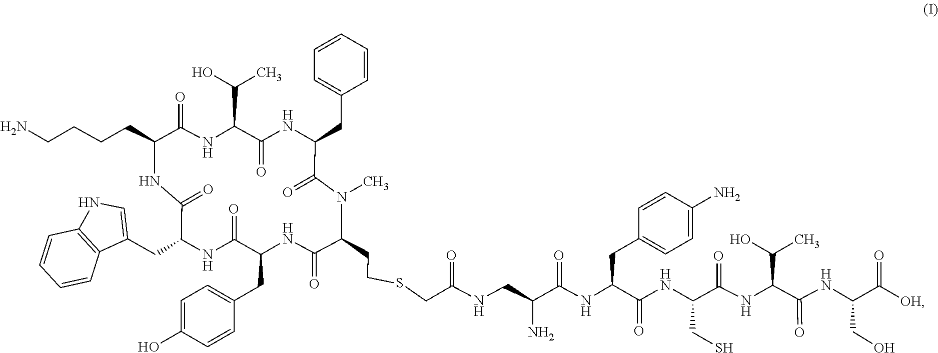 [SMILES: CC(O)[C@H](NC(=O)[C@H](CS)NC(=O)[C@H](Cc1ccc(N)cc1)NC(=O)[C@@H](N)CNC(=O)CSCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N[C@@H](Cc2ccccc2)C(=O)N1C)C(=O)N[C@@H](CO)C(=O)O]

or a pharmaceutically acceptable salt thereof, and an effective amount of a reducing agent to affect chelation of the compound of formula I with ¹⁸⁶Re and/or ¹⁸⁸Re;

(ii) a second vial containing effective stabilizing amounts of gentisic acid and ascorbic acid; and
(iii) a third vial containing a predetermined quantity of a histone deacetylase inhibitor.

In an embodiment, the HDACi is selected from the group consisting of hydroxamic acids, short chain fatty acids, benzamides, cyclic tetrapeptides, sirtuins inhibitors and combinations thereof. In an embodiment, the HDACi is a short chain fatty acid.

In an embodiment, the HDACi is selected from the group consisting of Trichostatin A, SAHA, Belinostat, Panabiostat, Givinostat, Resminostat, Abexinostat, Quisinostat, Rocilinostat, Practinostat, CHR-3996, valproic acid, butyric acid, phenylbutyric acid, Entinostat, Tacedinaline, 4SC202, Mocetinostat, Romidepsin, nicotinamide, Sirtinol, Cambinol, EX-527 and combinations thereof. In an embodiment, the HDACi is selected from the group consisting of valproic acid, butyric acid, phenylbutyric acid and combinations thereof. In an embodiment, the HDACi is valproic acid.

In an embodiment, the quantity of a compound having formula I in the first vial is from 45 μg to 60 μg of anhydrous material. In an embodiment, the quantity of a compound having formula I in the first vial is about 70 μg of P2045 trifluoroacetate.

In an embodiment, the reducing agent is SnCl₂. In an embodiment, the amount of SnCl₂ is about 850 μg.

In an embodiment, the gentisic acid in the second vial is in the form of gentisic acid, sodium salt, monohydrate. In an embodiment, the quantity of gentisic acid, sodium salt, monohydrate is about 20 mg. In an embodiment, the quantity of ascorbic acid in the second vial is about 10 mg.

In an embodiment, the predetermined amount of histone deacetylase inhibitor in the third vial is 250 mg.

### BRIEF DESCRIPTION OF THE DRAWINGS

FIG. 1 shows a tumor regression study in NCI-H69 mice treated with ¹⁸⁸Re-P2045.

FIG. 2 shows gel chromatographs indicating upregulation of SSTR2 in small cell cell lines after 24 hours of incubation with 3 and 5 mM valproic acid versus beta-actin (control).

FIG. 3 graphically quantifies the expression of SSTR2 in small cell cell lines after 24 hours of incubation with 3 and 5 mM valproic acid versus the demethylating agent, Decitabine.

### DETAILED DESCRIPTION

Small cell lung cancer now accounts for approximately 10-15% of the 225,000 cases of lung cancer in the United States. Patients with malignant effusions or distant metastases (i.e. stage IV or “extensive” disease) account for 65% of newly diagnosed cases and have a median survival of approximately 12 months with current therapy and essentially no three-year survival. Patients with limited disease (stage Ia-IIIb) account for less than 35% of cases and 75% have an overall median survival of less than 24 months. Treatment of advanced SCLC consists of initial chemotherapy with a platinum agent (cisplatin or carboplatin) and etoposide. Topotecan (either oral or IV) is approved for relapses occurring greater than 60 days after initial therapy. Despite numerous trials, there has not been a significant advance in treatment of SCLC in over 20 years and virtually all patients with metastases will ultimately succumb to the disease.

The incidence of malignant neuroendocrine tumors (NETs) has been steadily increasing from 1.09 new cases per 100,000 in 1973 (age-adjusted incidence) to 5.25 per 100,000 in 2004 based on the US survey data from the Surveillance, Epidemiology, and End Results (SEER) program and more recent population based studies.

Extrapulmonary small cell lung carcinoma (EPSCLC) is a rare malignancy that can occur in virtually any organ. Approximately 1000 cases are diagnosed/year in the United States. They are generally treated in a manner similar to SCLC and have a generally poor prognosis with an estimated 5-year survival of less than 15%.

Large cell neuroendocrine carcinoma (LCNECs) is a controversial entity that is formally considered a non-small cell lung cancer, however, it is frequently treated as SCLC and shares several biological features. It accounts for approximately 9% of all lung cancers (about 20,000 cases/year). In general, these patients fare worse than other NSCLC patients, with a 5-year survival rate of 15-57%, depending upon stage.

Many of these tumors are functional and produce a variety of peptide hormones, leading to distressing symptoms including flushing, diarrhea, hypoglycemia and others. Localized disease can be resected for cure. However, a minority of patients present with inoperable (usually metastatic disease) for which a curative procedure is not feasible and/or possible. For inoperable symptomatic patients, the mainstay of therapy has been long acting somatostatin analogues. The responsiveness, both symptomatically (in hormone producing tumors) and in terms of radiologic response and survival provides strong supporting evidence for the use of somatostatin targeted agents [Rinke et al., ‘Placebo-controlled, double-blind, prospective, randomized study on the effect of octreotide LAR in the control of tumor growth in patients with metastatic neuroendocrine midgut tumors: A report from the PROMID Study Group,’ Journal of Clinical Oncology (2009), Volume 27, Pages 4656-4663; Caplin et al., ‘A randomized, double-blind, placebo-controlled study of lanreotide: antiproliferative response in patients with gastroenteropancreatic neuroendocrine tumors (CLARINET),’ Abstract, 2013 European Cancer Congress]. Though the expression level is frequently less seen in well differentiated NE tumors, all of these cancers commonly express SSTR2 receptors and therefore may benefit from SSTR2 directed therapy [Maecke et al., ‘Somatostatin receptors as targets for nuclear medicine imaging and radionuclide treatment,’ Journal of Nuclear Medicine (2011), Volume 52(6), Pages 841-844; Kwekkeboom et al., ‘Radioiodinated somatostatin analog scintigraphy in small-cell lung cancer,’ Journal of Nuclear Medicine (1991), Volume 32(10), Pages 1845-1848; Fujita et al., ‘Gene expression of somatostatin receptor subtypes, SSTR1 and SSTR2, in human lung cancer cell lines,’ Life Sciences (1994), Volume 55, Pages 1797-1806; O'Byrne et al., ‘Somatostatin, its receptor and analogs in lung cancer,’ Chemotherapy (2001), Volume 47 (suppl 2), Pages 78-108]. Numerous studies, done primarily in Europe, have demonstrated that various SSTR2 targeted radiopharmaceuticals produce meaningful responses, both radiologically and symptomatically in SSTR2 expressing malignancies [Zaknun et al., “The joint IAEA, EANM, and SNMMI practical guidance on peptide receptor radionuclide therapy (PRRNT) in neuroendocrine tumours,” European Journal of Nuclear Medicine and Molecular Imaging (2013), Volume 40(5), Pages 800-816].

In addition, valproic acid has recently been investigated as an anticancer agent, alone and in combination with chemotherapeutics. [Brode and Brades, “Could valproic acid be an effective anticancer agent? The evidence so far,” Expert Rev Anticancer Ther (2014 October), Volume 14(10), Pages 1097-1100; Eckschlager et al., “Histone Deacetylase Inhibitors as Anticancer Drugs,” International Journal of Molecular Sciences, (2017), Volume 18, Page 1414; Tsai et al., “Valproic acid suppresses cervical cancer tumor progression possibly via activating Notch1 signaling and enhances receptor-targeted cancer chemotherapeutic via activating somatostatin receptor type II,” Arch Gynecol Obstet (Published online 23 Feb. 2013)].

The SSTR2-binding ^{99m}Tc-labeled peptide Depreotide (NeoTect®) has been approved for detection of somatostatin receptor (SSTR)-expressing pulmonary masses [Menda and Kahn, ‘Somatostatin receptor imaging in non-small cell lung cancer with ^{99m}Tc-Depreotide,’ Seminars in Nuclear Medicine (2002) Volume 32, pages 92-96; Kumar et al., ‘Somatostatin receptors in primary human breast cancer: quantitative analysis of mRNA for subtypes 1-5 and correlation with receptor protein expression and tumor pathology,’ Breast Cancer Research and Treatment (2005) Volume 92(2), pages 175-186]. Depreotide is a peptidyl agent having a cyclic hexapeptide portion that binds to the somatostatin receptor and a tetrapeptide portion that chelates the radioactive element ^{99m}Tc.

SSTRs and in particular, the type 2 SSTR (SSTR2) are commonly expressed in a variety of neuroendocrine tumors including carcinoids, GI neuroendocrine tumors and both small cell and non-small cell lung cancer. SSTRs are also expressed on peritumoral blood vessels in several malignancies, raising the possibility of vascular targeting [Dizeyi et al., ‘Localization and mRNA expression of somatostatin receptor subtypes in human prostatic tissue and prostate cancer cell lines,’ Urologic Oncology (2002) Volume 7(3), pages 91-98]. Importantly, there is relatively low expression of SSTR2 on normal tissues, thus providing the potential for a good therapeutic index should a somatostatin receptor-binding moiety be attached to a radiotherapeutic isotope.

Accordingly, a method of treating a somatostatin subtype 2-expressing cancer in a patient comprises administering an effective amount of a compound having the formula I:

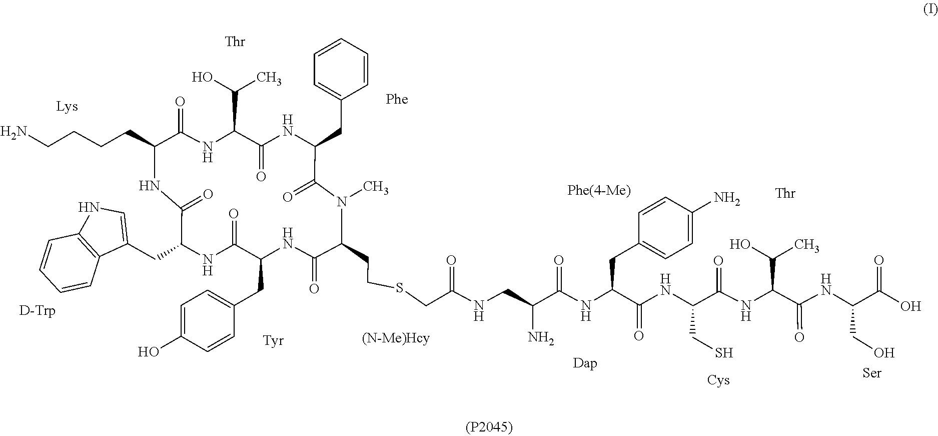 [SMILES: CC(O)[C@H](NC(=O)[C@H](CS)NC(=O)[C@H](Cc1ccc(N)cc1)NC(=O)[C@@H](N)CNC(=O)CSCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N[C@@H](Cc2ccccc2)C(=O)N1C)C(=O)N[C@@H](CO)C(=O)O]

or a pharmaceutically acceptable salt thereof, wherein the compound of formula I is bonded to a radiotherapeutic isotope of rhenium to form a rhenium chelate; and (ii) an effective amount of topotecan, or a pharmaceutically acceptable salt thereof; or
an effective amount of a histone deacetylase inhibitor, or a pharmaceutically acceptable salt thereof.

The compound of formula I (hereafter P2045) is an 11 amino acid peptide cyclo-[Tyr-(D-Trp)-Lys-Thr-Phe-(N-Me)Hcy]CH₂CO-β-Dap-Phe(4-NH₂)-Cys-Thr-Ser-OH)], where ‘Hcy’ is homocysteine and ‘Dap’ is diaminopropionic acid.

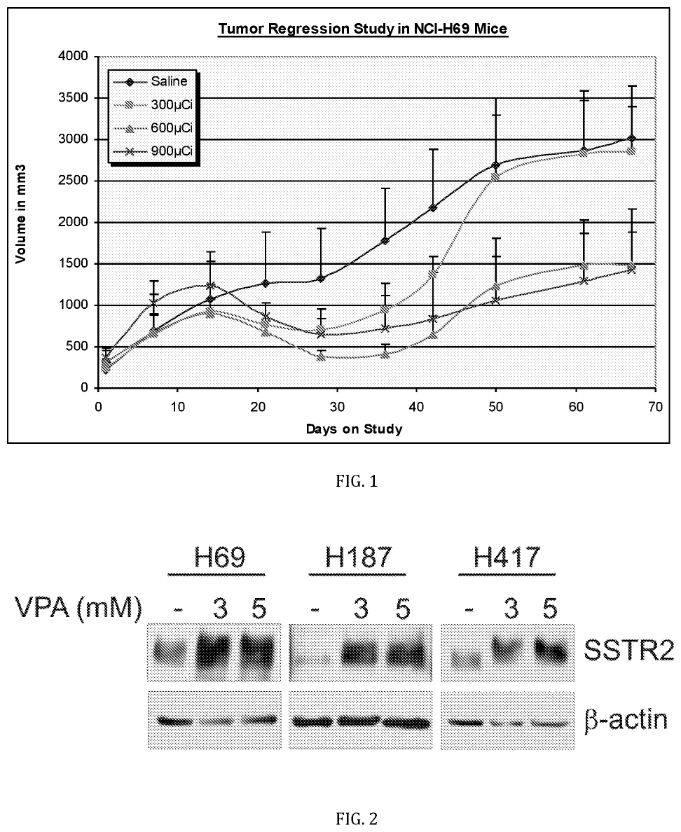

Radiolabeled with radioactive isotopes of rhenium, P2045 retains the SSTR affinity of Depreotide but with lower kidney and whole body retention. Rhenium-186 (¹⁸⁶Re) is a beta-emitting isotope with a half-life of 90 hours, maximum particle energy of 1.08 MeV, and a path length of approximately 4.5 mm. Rhenium-188 (¹⁸⁸Re) is a beta-emitting isotope with a half-life of 17 hours, maximum particle energy of 2.1 MeV, and a path length of approximately 5 mm. In studies with nude mice, ¹⁸⁸Re-P2045 caused almost complete inhibition of SSTR-bearing pancreatic tumors with no acute damage on histopathologic examination to any other organ or tissue [Cyr et al., ‘Somatostatin receptor-binding peptides suitable for tumor radiotherapy with Re-188 or Re-186: Chemistry and Initial Biolgical Studies,’ Journal of Medicinal Chemistry (2007), Volume 50, Pages 1354-1364]. In another study, evaluation of ¹⁸⁸Re-P2045 demonstrated significant growth delay in a small cell lung cancer model at higher single doses of the agent. See FIG. 1.

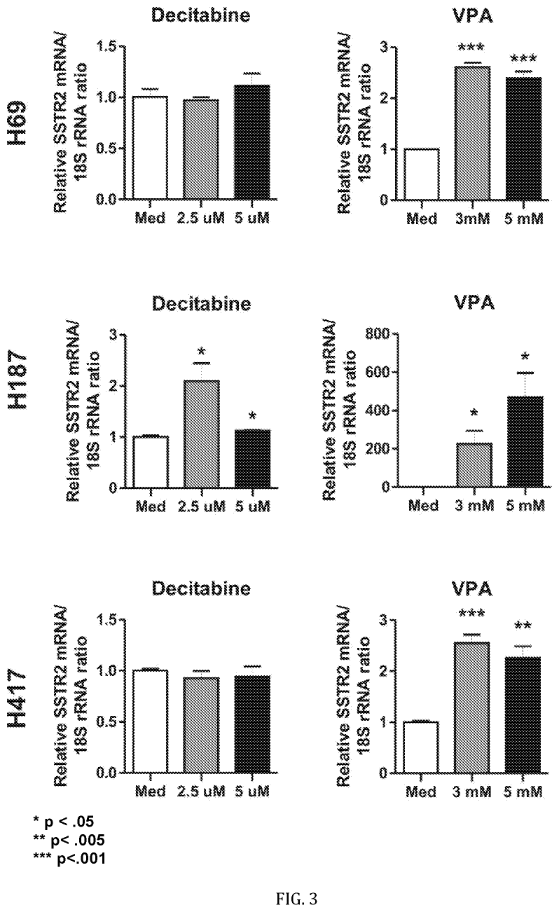

FIG. 2 shows gel chromatographs indicating upregulation of SSTR2 in small cell cell lines (H69, H187 and H417) after 24 hours of incubation with 3 and 5 mM valproic acid versus beta-actin (control). In contrast, similar experiments done with the demethylating agent, Decitabine, failed to demonstrate upregulation of SSTR2 in two of the three tested cell lines. FIG. 3 graphically quantifies the expression of SSTR2 and Decitabine. These data demonstrate the potential of a simple, inexpensive pharmacologic maneuver to upregulate SSTR2 receptors and therefore, enhance and broaden the activity of SSTR2 targeted treatments.

The structure of P2045 chelated to a radiotherapeutic metal is shown in a compound of formula II:

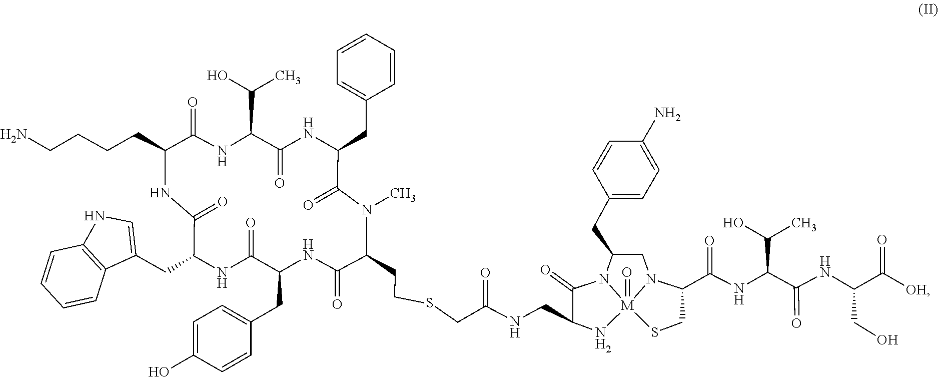

where M is ¹⁸⁶Re or ¹⁸⁸Re.

The P2045-chelated radiotherapeutic isotope of rhenium is administered to a patient having a cancer overexpressing SSTR2 intravenously one or more times (e.g., one, two, or three times), but no more than once within a 28-day period at a dose of from 80 mCi/m² to 250 mCi/m².

In an example, ¹⁸⁸Re-P2045 is prepared using a Kit for the Preparation of ¹⁸⁸Re-P2045: a two-vial, single-dose, lyophilized product. Vial 1 of the kit contains 70 μg of the trifluoroacetate salt of P2045 (equivalent to approximately 53 μg of anhydrous, counter-ion-free P2045); 25 mg of Sodium α-D-glucoheptonate dihydrate; 850 μg of tin (II) chloride dihydrate; and 100 μg of edetate disodium USP. Vial 2 of the kit contains 20 mg of gentisic acid, monosodium salt monohydrate and 10 mg of ascorbic acid USP.

Accordingly, ¹⁸⁸Re-sodium perrhenate 0.9% Sodium Chloride for Injection USP is made available to the clinical site on the morning of the patient injection for preparation of the dose, by either installing a W 188/Re 188 generator at the clinical site or by shipping ¹⁸⁸Re-sodium perrhenate from a regional radiopharmacy. Alternatively, ¹⁸⁸Re-P2045 can be prepared at a regional radiopharmacy and shipped to the clinical site on the morning of the patient injection.

Vial 1 of Kit for the Preparation of ¹⁸⁸Re-P2045 is reconstituted with 80±2 mCi ¹⁸⁸Re-sodium perrhenate in a maximum volume of 3 mL and heated in a boiling water bath for 15 minutes. Vial 1 is then removed from the boiling water bath and allowed to cool for 3 minutes in a refrigerated lead container. While Vial 1 is cooling, Vial 2 is reconstituted with a volume of 0.9% Sodium Chloride for Injection USP to produce a final volume of 5 mL (i.e., total volume of Vial 1 and Vial 2 combined). The necessary volume of ¹⁸⁸Re-P2045 solution is adjusted to produce the prescribed radioactive dose. The number of kits per patient will vary, depending on the dose level assigned and the patient body surface area (i.e., a dose of 80 mCi/m² to 250 mCi/m²).

In order to properly assess the need for patient treatment, prior to treatment with ¹⁸⁶Re-P2045 or ¹⁸⁸Re-P2045, the patient's tumor is imaged by administration of ^{99m}Tc-2045 (a compound of formula II where M is ^{99m}Tc) followed by gamma camera scintigraphy. Accordingly, the kit used for the preparation of ^{99m}Tc-2045 includes a one-vial, single dose, lyophilized product. The contents of the vial are contained in 5 mL USP Type I glass tubing vials with gray butyl rubber lyophilization stoppers sealed with aluminum crimp caps. Typically, the vial contains sodium alpha-D-glucoheptonate dihydrate, which serves as a bulking agent; stannous chloride dihydrate which serves to reduce ^{99m}Tc from the +7 oxidation state found in sodium pertechnitate to the +5 oxidation state favorable for binding to nitrogen and sulfur atoms. The kit may also contain Edetate Disodium USP to serve as an aid in radiolabeling.

^{99m}Tc-2045 is prepared in situ at the site of use by reconstituting a Kit for the Preparation of ^{99m}Tc-2045 drug product with ^{99m}Tc-Sodium Pertechnetate Injection USP. Following reconstitution, the kit solution is incubated at room temperature for 30 minutes and analyzed for radiochemical purity by instant thin layer chromatography (ITLC). ^{99m}Tc-2045 is normally stable (>1=85% radiochemical purity by ITLC) for at least 5 hours after preparation when stored at room temperature.

Subsequent to the administration of ^{99m}Tc-2045 and subsequent assessment of tumor burden by scintigraphy, but prior to the administration of ¹⁸⁶Re-2045 or ¹⁸⁸Re-2045, the patient is treated with topotecan. Topotecan is an anti-tumor drug with topoisomerase I inhibitory activity. Intravenous (IV) topotecan is marketed in over 70 markets worldwide, including the European Medicines Evaluation Agency (EMEA) Committee for Proprietary Medicinal Products (CPMP) and the U.S. Food and Drug Administration (FDA) for the treatment of metastatic carcinoma of the ovary after failure of initial or subsequent chemotherapy. Intravenous topotecan has also been approved by the FDA and in over 30 countries worldwide for the treatment of small-cell lung cancer after failure of first-line chemotherapy. Topotecan was also recently approved by the FDA for the treatment of cervical cancer. The most common toxicities for topotecan observed when it is administered intravenously at the dose of 1.5 mg/m², daily×5, every 21 days, are hematologic (grade 4 neutropenia, thrombocytopenia and grade 3/4 anemia). Dalezis et al., ‘Combinational effect of topotecan and octreotide on murine leukemia cells in vivo and in vitro,’ Journal of the Balkan Union of Oncology (2006), Volume 11(3), Pages 323-327.

Topotecan is commercially available and is supplied in vials as a light yellow, lyophilized cake. Each vial contains 4 mg of topotecan. Other ingredients in the formulation include 48 mg mannitol, 20 mg tartaric acid and NaOH/HCl (to adjust pH to 3.0). The lyophilized formulation of topotecan is reconstituted with 4 mL of sterile water for injection prior to dilution with 5% dextrose solution suitable for injection or 0.9% sterile saline solution. Because the lyophilized dosage form contains no antibacterial preservatives, the reconstituted (undiluted) is discarded 24 hours after initial reconstitution. Final dilution with buffered solutions must be between 10 mcg/mL and 50 mcg/mL to ensure stability.

Reconstituted topotecan can be administered intravenously over a 30-minute period. Individual patient dose is based on body surface area calculated from height and actual weight using the nomogram derived from the Dubois and Dubois formula. The lyophilized formulation must be reconstituted with 4 ml of Sterile Water for Injection prior to dilution with 0.9% Saline Solution or 5% Dextrose Injection.

In the present formulations, topotecan is administered intravenously at a daily dose of from 1 mg/m² to 1.5 mg/m² four, five, or six days before the administration of ¹⁸⁶Re-2045 or ¹⁸⁸Re-2045. Additional topotecan can be administered up to three times during the 28-day period after administration of ¹⁸⁶Re-2045 or ¹⁸⁸Re-2045.

Alternatively, instead of topotecan the patient is treated with valproic acid, or another histone deacetylase inhibitor, before treatment with ¹⁸⁶Re-2045 and/or ¹⁸⁸Re-2045. Valproic acid (Depakene™) is a carboxylic acid having a molecular weight of 144 and occurring as a colorless liquid with a characteristic odor. It is slightly soluble in water (1.3 mg/mL) and very soluble in organic solvents. Valproic acid capsules and syrup are commonly administered orally for the treatment of petit mal, grand mal, mixed and akinetic-myoclonic seizures. Each soft elastic capsule typically contains 250 mg valproic acid. The syrup typically contains the equivalent of 250 mg valproic acid per 5 mL as the sodium salt. Inactive ingredients in either capsule or syrup form include corn oil, FD&C Yellow No. 6, gelatin, glycerin, iron oxide, methylparaben, propylparaben, and titanium dioxide. In the present invention, valproic acid is administered orally at a daily dose of from 75 mg/kg to 150 mg/kg, where the daily dose is divided into 2 or 3 portions and is administered over 7 days.

The patient is optionally administered amino acids and/or electrolytes before treatment with ¹⁸⁶Re-2045 and/or ¹⁸⁸Re-2045. Examples of amino acid infusion solutions include Aminosyn®, Aminosyn®II, FreAmine®, FreAmine®II, FreAmine®III, and Travasol®. The administration is typically given intravenously through a peripheral vein with a dilute (5 to 10%) dextrose solution.

### Definitions

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present formulations also include pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of compounds used in compositions of the present invention include the conventional non-toxic salts or the quaternary ammonium salts of the parent compound formed, for example, from non-toxic inorganic or organic acids. The pharmaceutically acceptable salts of compounds used in compositions of the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22nd edition, Pharmaceutical Press, 2012. Pharmaceutically acceptable solvates are also described therein.

As used herein, the term “person” or “patient” or “subject” used interchangeably, refers to a human, or to any animal, including mammals, such as mice, rats, other rodents, rabbits, dogs, cats, swine, cattle, sheep, horses, or primates.

As used herein, the phrase “therapeutically effective amount” refers to the amount of a pharmaceutical composition, or of an individual compound in a pharmaceutical composition, that elicits the biological or medicinal response in a tissue, system, animal, individual or human that is being sought by a researcher, veterinarian, medical doctor or other clinician, which includes one or more of the following: (i) treating proliferative disease in a person who is experiencing or displaying the pathology or symptomotology related to cancer (i.e., arresting further development of the pathology and/or symptomotology) such as stabilizing or decreasing tumor load or volume; and (ii) ameliorating proliferative disease; for example, ameliorating a condition or disorder in a person who is experiencing or displaying the pathology or symptomotology of proliferative disease, condition or disorder (i.e., reversing the pathology and/or symptomotology) such as lowering the rate of tumor growth or metastatic progression.

As used herein, the term “about” means+/−10% of the indicated value.

### Pharmaceutical Formulations and Dosage Forms

The combinations of compounds in the pharmaceutical compositions disclosed herein may conveniently be presented for use in the form of a pharmaceutical formulation and thus pharmaceutical formulations comprising a combination of compounds as defined above together with a pharmaceutically acceptable carrier are contemplated by the present disclosure. The individual components of such combinations may be administered either sequentially or simultaneously in separate pharmaceutical formulations. These pharmaceutical compositions can be prepared in a manner well known in the pharmaceutical arts.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the composition preparations typically will be between 3 and 11, or from 5 to 9, or from 7 to 8. It will be understood that use of certain of the foregoing excipients, carriers, or stabilizers will result in the formation of pharmaceutical salts.

### Kits

The present invention also includes pharmaceutical kits (also referred to as packages) useful, for example, in the treatment or prevention of cancer, which include two or more containers containing the pharmaceutical agents of the compositions of the invention, optionally in combination with at least one pharmaceutically acceptable carrier, and together comprising a therapeutically effective amount of a composition of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Example 1. Treatment of a Non-Small Cell Lung Carcinoma Patient with a ¹⁸⁸Re-P2045/Topotecan Combination

In order to assess the extent of disease and to estimate the radiation absorbed dose that would result from treatment with ¹⁸⁸Re-P2045, the patient is first treated with ^{99m}Tc-P2045 and imaged for tumor uptake of this agent using a gamma camera and a standard imaging protocol such as, for example, single photon emission computed tomography (SPECT) for whole body or regional imaging. See Herman, Fundamentals of Computerized Tomography: Image Reconstruction from Projections, 2nd ed., (2009), published by Springer. Within 30 minutes prior to administration, vital signs (pulse, respiratory rate, blood pressure, and temperature) are obtained. A pre-prepared vial containing P2045, Sodium a-D-Glucoheptonate Dihydrate, Tin (II) Chloride, and Edetate Disodium USP (see Table 1) was reconstituted with Sodium Pertechnetate ^{99m}Tc for Injection (35±7 mCi in a maximum volume of 1 mL) and incubated at room temperature for 30 minutes. Following incubation, the volume of the kit is adjusted to 3 mL with 0.9% Sodium Chloride for Injection and the entire content of the kit filtered through a 0.22 micron filter and transferred in a sterile syringe for intravenous injection to the patient. Quality control testing of the final preparation to determine radiochemical purity is performed using instant thin layer chromatography (ITLC) strips. If the radiochemical purity is less than 85%, the ^{99m}Tc-P2045 preparation is not used. The amount of product administered to the patient is adjusted to deliver the desired radioactive dose (20 to 25 mCi). The syringe containing ^{99m}Tc-P2045 is assayed in a dose calibrator immediately before injection. The IV line is kept in place for at least one hour following administration of study drug, to provide access in case an acute adverse event requires intravenous medication therapy. Following removal of the IV line, the IV catheter and tubing is assayed in the same dose calibrator for residual activity along with the empty syringe. Post-injection vital signs are taken at the end of injection and 1 hour after injection.

TABLE 1
Composition of the Kit for the Preparation of ^{99m}Tc-P2045

| Component | Quantity per vial | |
|---|---|---|
| P2045 Peptide Trifluoroacetate | 70 | μg¹ |
| Sodium α-D-Glucoheptonate Dihydrate | 25 | mg |
| Tin (II) Chloride Dihydrate ACS | 50 | μg |
| Edetate Disodium USP² | 100 | μg |

¹Corresponds to approximately 53 μg of anhydrous, counter-ion-free P2045 Peptide
²Edetate Disodium USP is the dihydrate form of disodium edetate
The vial is adjusted to pH 7.4 ± 0.1 with sodium hydroxide and/or hydrochloric acid prior to lyophilization.

Following treatment with ^{99m}Tc-P2045 and subsequent analysis the dosing schedule for the administration of ¹⁸⁸Re-P2045 and topotecan is followed as indicated in Table 2 and as described below.

TABLE 2
Treatment Regimen for administration
of ¹⁸⁸Re-P2045 and topotecan

| | | | | | Cycle |
|---|---|---|---|---|---|
| Agent | Premedication | Dose | Route | Schedule | Length |
| ¹⁸⁸Re-P2045 | Amino acid | From 80 | IV | once | 28 |
| | infusion | mCi/m² to | | | days |
| | | 250 mCi/m² | | | |
| Topotecan | Antiemetics | From | IV | up to 3 | |
| | (decadron, | 1-1.5 mg/m² | | times | |
| | 5HT3 | daily x 3 IV | | | |
| | antagonist or | | | | |
| | equivalent) | | | | |

Prior to the administration of topotecan, the patient is treated with dexamethasone or an antiemetic such as a 5HT3 antagonist. Examples of 5HT3 antagonists include dolasetron and palonosetron.

Topotecan is then administered to the patient via intravenous (IV) infusion at a dose of from 1 mg/m² to 1.5 mg/m². The patient is followed for the advent of any adverse reactions and then, 4 days after the administration of topotecan, ¹⁸⁸Re-P2045 is administered.

Prior to administration of ¹⁸⁸Re-P2045, the patient is optionally treated with an infusion of an amino acid/electrolyte solution such as Aminosyn® II (Abbott). If nausea and vomiting occur during or after the amino acid infusion, the patient is treated with appropriate antiemetics, which can include steroids, olanzapine, metoclopramide and/or prochlorperazine as determined by the treating physician.

Four to twenty four hours after treatment with the amino acid/electrolyte solution, the patient is given ¹⁸⁸Re-P2045 via IV administration at a dose of from 80 mCi/m² to 250 mCi/m². ¹⁸⁸Re-P2045 is prepared using the Kit for the Preparation of ¹⁸⁸Re-P2045, a two-vial, single-dose, lyophilized product. Table 3 presents the composition of the two vials.

Accordingly, ¹⁸⁸Re sodium perrhenate in 0.9% sodium chloride for injection USP is produced from a ¹⁸⁸W/¹⁸⁸Re generator. Vial 1 of the Kit for the Preparation of ¹⁸⁸Re-P2045 is reconstituted with 80±2 mCi of generator-produced sodium perrhenate in a maximum volume of 3 mL and heated in a boiling water bath for 15 minutes. Vial 1 is removed from the boiling water bath and allowed to cool for 3 minutes in a refrigerated lead container. While Vial 1 is cooling, the Kit for the Preparation of ¹⁸⁸Re-P2045 Vial 2 is reconstituted with the enough 0.9% sodium chloride for injection USP to produce a final volume of 5 mL (i.e., total volume of Vial 1 and Vial 2 combined). The volume of the resulting ¹⁸⁸Re-P2045 solution is then adjusted to produce the prescribed radioactive dose and filtered through a 0.22 micron filter to ensure sterility. Quality control testing of the final preparation to determine radiochemical purity is performed using instant thin layer chromatography (ITLC) strips. If the radiochemical purity is less than 90%, the ¹⁸⁸Re-P2045 preparation is not used. The number of kits used per patient is determined by the dose level assigned as determined by ^{99m}Tc-P2045 imaging and by patient body surface area.

TABLE 3
Composition of Vials 1 and 2 of the Kit
for the Preparation of ¹⁸⁸Re-P2045

| | |
|---|---|
| Component | Quantity per vial |

Composition of Lyophilized Vial 1

| | | |
|---|---|---|
| P2045 Peptide Trifluoroacetate | 70 | μg¹ |
| Sodium α-D-Glucoheptonate Dihydrate | 25 | mg |
| Tin (II) Chloride Dihydrate ACS | 850 | μg |
| Edetate Disodium USP² | 100 | μg |

Composition of Lyophilized Vial 2

| | | |
|---|---|---|
| Gentisic Acid Sodium Salt Monohydrate | 20 | mg |
| Ascorbic Acid USP² | 10 | mg |

¹Corresponds to approximately 53 μg of anhydrous, counter-ion-free P2045 Peptide
²Edetate Disodium USP is the dihydrate form of disodium edetate
Each vial is adjusted to pH 7.4 ± 0.1 with sodium hydroxide and/or hydrochloric acid prior to lyophilization.

The following whole body imaging standard is used in order to estimate the radiation absorbed doses of ¹⁸⁸Re-P2045: (i) Mark a 500-mL plastic bottle as “Imaging Standard Bottle”; (ii) Add 500-mL 0.9% sodium chloride solution in the container; (iii) Transfer a 200 μL aliquot of the dose into a centrifugal tube; (iv) Assay the tube in the dose calibrator and record radioactivity and time of assay; (v) Pipette out 150 μL into the “Imaging Standard” bottle; (vi) Rinse pipette tip several times with the “Imaging Standard” solution; (vii) Eject the pipette tip in a 2-in wide tape and wrap the tip in tape; (viii) Close “Imaging Standard” bottle and secure in a Zip-Lock plastic bag; (ix) Assay the tube in the dose calibrator and record the radioactivity and time of assay; and (x) Assay the wrapped pipette tip in the dose calibrator and record the radioactivity and time of assay.

Simultaneous anterior and posterior whole body imaging is optionally performed at approximately 1, 4.5, 24 and 48 hours following the administration of ¹⁸⁸Re-P2045. Patients are instructed not to void before the 1-hour scan is completed but should void prior to the 4.5, 24 and 48 hour sessions. Patients must remain well-hydrated for 48 hours post-administration. Duration of the imaging session may vary and is dependent upon the dose of ¹⁸⁸Re-P2045 administered.

After a period of 7 days subsequent to the administration of ¹⁸⁸Re-P2045, topotecan is administered once more according to the procedure described above. After another wait period of 7 days, topotecan is administered once more as described above for a total of three topotecan administrations, each at a dose of from 1 mg/m² to 1.5 mg/m².

### Example 2. Treatment of a Non-Small Cell Lung Carcinoma Patient with a ¹⁸⁸Re-P2045/Valproic Acid Combination

In order to assess the extent of disease and to estimate the radiation absorbed dose that would result from treatment with ¹⁸⁸Re-P2045, the patient is first treated with ^{99m}Tc-P2045 and imaged for tumor uptake of this agent as described in Example 1.

Following administration of ^{99m}Tc-P2045 and subsequent analysis, the dosing schedule for the administration of ¹⁸⁸Re-P2045 and valproic acid is followed as indicated in Table 4 and as described below.

TABLE 4
Treatment Regimen for administration
of ¹⁸⁸Re-P2045 and valproic acid

| | | | | | Cycle |
|---|---|---|---|---|---|
| Agent | Premedication | Dose | Route | Schedule | Length |
| ¹⁸⁸Re-P2045 | Amino acid | From 80 | IV | once | 28 |
| | infusion | mCi/m² to | | | days |
| | | 250 mCi/m² | | | |
| Depakene ™ | none | From | oral | over 1 day | |
| | | 75 mg/kg to | | | |
| | | 150 mg/kg | | | |

Depakene™ (valproic acid) capsules and syrup are antiepileptics for oral administration. Each soft elastic capsule contains 250 mg valproic acid. The syrup contains the equivalent of 250 mg valproic acid per 5 mL as the sodium salt. Inactive Ingredients 250 mg capsules: corn oil, FD&C Yellow No. 6, gelatin, glycerin, iron oxide, methylparaben, propylparaben, and titanium dioxide. Oral Solution: FD&C Red No. 40, glycerin, methylparaben, propylparaben, sorbitol, sucrose, water, and natural and artificial flavors. Accordingly, Depakene™ (valproic acid), as a capsule or syrup, is administered to the patient at least 24 hours prior to the administration of ¹⁸⁸Re-P2045. The administration of the dosage given in Table 4 can be either b.i.d. or t.i.d.

After administration of valproic acid but prior to administration of ¹⁸⁸Re-P2045, the patient is optionally treated with an infusion of an amino acid/electrolyte solution such as Aminosyn® II (Abbott). If nausea and vomiting occur during or after the amino acid infusion, the patient is treated with appropriate antiemetics, which can include steroids, olanzapine, metoclopramide and/or prochlorperazine as determined by the treating physician.

Four to twenty four hours after treatment with the amino acid/electrolyte solution, the patient is given ¹⁸⁸Re-P2045 via IV administration at a dose of from 80 mCi/m² to 250 mCi/m². ¹⁸⁸Re-P2045 is prepared using the Kit for the Preparation of ¹⁸⁸Re-P2045, a two-vial, single-dose, lyophilized product, as indicated in Example 1 and Table 3, and subsequent imaging is performed as indicated in Example 1.

While the invention has been described in detail and with reference to specific examples thereof, it will be apparent to one skilled in the art that various changes and modifications can be made therein without departing from the spirit and scope thereof.

It is appreciated that certain features of the invention, which are, for clarity, described in the context of separate embodiments, can also be provided in combination in a single embodiment. Conversely, various features of the invention which are, for brevity, described in the context of a single embodiment, may also be provided separately or in any suitable subcombination.

### STATEMENTS REGARDING INCORPORATION BY REFERENCE AND VARIATIONS

All references cited throughout this application, for example patent documents including issued or granted patents or equivalents; patent application publications; and non-patent literature documents or other source material; are hereby incorporated by reference herein in their entireties, as though individually incorporated by reference.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments, exemplary embodiments and optional features, modification and variation of the concepts herein disclosed can be resorted to by those skilled in the art, and that such modifications and variations are considered to be within the scope of this invention as defined by the appended claims. The specific embodiments provided herein are examples of useful embodiments of the invention and it will be apparent to one skilled in the art that the invention can be carried out using a large number of variations of the devices, device components, and method steps set forth in the present description. As will be apparent to one of skill in the art, methods and devices useful for the present methods and devices can include a large number of optional composition and processing elements and steps. All art-known functional equivalents of materials and methods are intended to be included in this disclosure.

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure.

It must be noted that as used herein and in the appended claims, the singular forms “a”, “an”, and “the” include plural reference unless the context clearly dictates otherwise. Thus, for example, reference to “a tumor” includes a plurality of such tumors and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range and all the integer values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is/are not specifically disclosed herein.

1. A method for the treatment of a somatostatin subtype 2-expressing cancer in a patient comprising administering to said patient:
(i) an effective amount of a compound having the formula I: 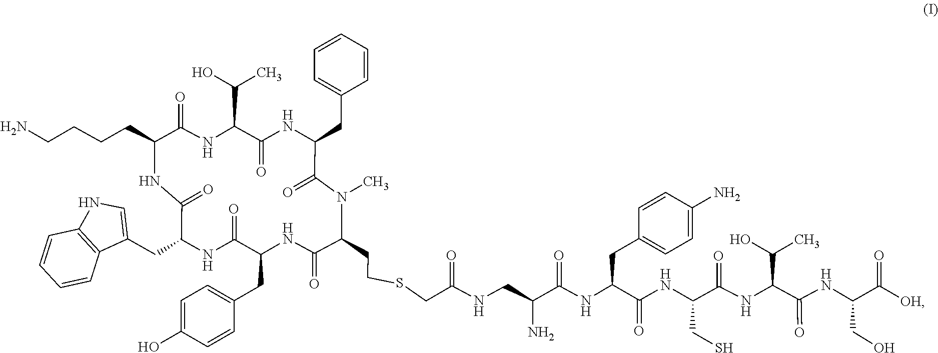 [SMILES: CC(O)[C@H](NC(=O)[C@H](CS)NC(=O)[C@H](Cc1ccc(N)cc1)NC(=O)[C@@H](N)CNC(=O)CSCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N[C@@H](Cc2ccccc2)C(=O)N1C)C(=O)N[C@@H](CO)C(=O)O]
or a pharmaceutically acceptable salt thereof, wherein said compound of formula I is bonded to a radiotherapeutic isotope of rhenium to form a rhenium chelate; and
(ii) an effective amount of topotecan, or a pharmaceutically acceptable salt thereof; or
an effective amount of a histone deacetylase inhibitor (HDACi), or a pharmaceutically acceptable salt thereof.

2. The method according to claim 1, wherein the HDACi is selected from the group consisting of hydroxamic acids, short chain fatty acids, benzamides, cyclic tetrapeptides, sirtuins inhibitors and combinations thereof.

3. The method according to claim 1, wherein the HDACi is selected from the group consisting of Trichostatin A, SAHA, Belinostat, Panabiostat, Givinostat, Resminostat, Abexinostat, Quisinostat, Rocilinostat, Practinostat, CHR-3996, valproic acid, butyric acid, phenylbutyric acid, Entinostat, Tacedinaline, 4SC202, Mocetinostat, Romidepsin, nicotinamide, Sirtinol, Cambinol, EX-527 and combinations thereof.

4. The method according to claim 1, wherein the HDACi is selected from the group consisting of valproic acid, butyric acid, phenylbutyric acid and combinations thereof.

5. The method according to claim 1, wherein said rhenium chelate is a compound having the formula II: 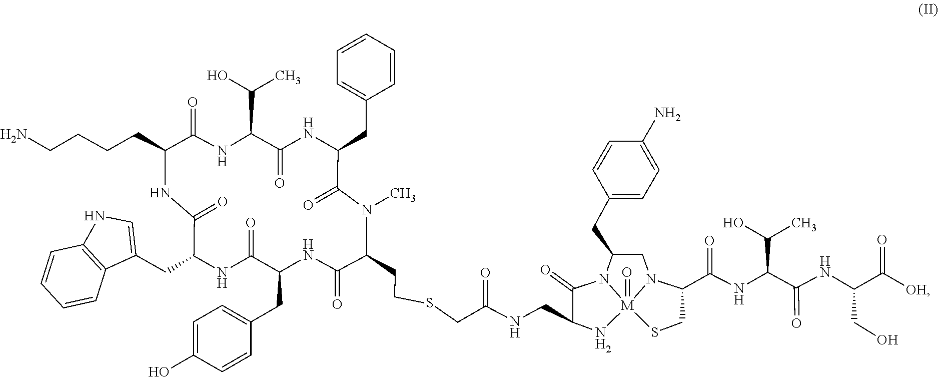
wherein M is said radiotherapeutic isotope of rhenium.

6. The method according to claim 1, wherein said radiotherapeutic isotope of rhenium is ¹⁸⁶Re and/or ¹⁸⁸Re.

7. The method according to claim 1, wherein said radiotherapeutic isotope of rhenium is administered at a dose of from 80 mCi/m² to 250 mCi/m².

8. The method according to claim 1, wherein said radiotherapeutic isotope of rhenium is administered intravenously no more than once within a 28-day period.

9. The method according to claim 1, further comprising administering a technetium chelate having formula II: 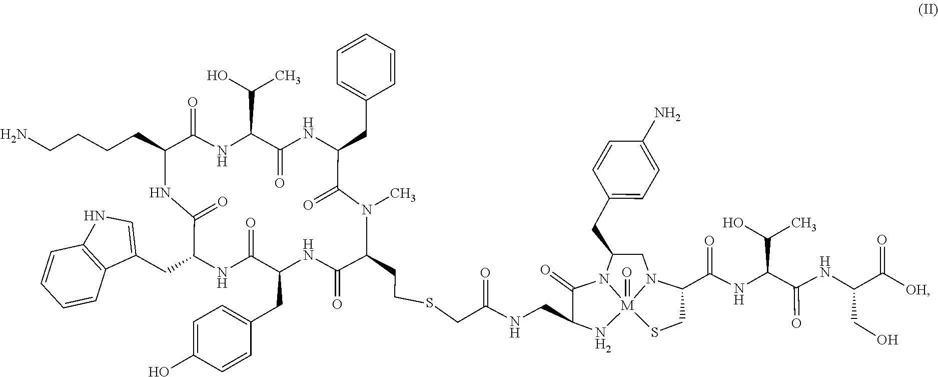
wherein M is ^{99m}Tc and said technetium chelate is administered before the administration of said rhenium chelate.

10. The method according to claim 9, wherein said technetium chelate is administered no more than 14 days before the administration of said rhenium chelate.

11. The method according to claim 1, further comprising administering a supplemental amino acid solution intravenously 4 hours to 24 hours prior to administering said rhenium chelate.

12. The method according to claim 11, wherein said amino acid solution comprises lysine.

13. The method according to claim 1, wherein said effective amount of topotecan is administered intravenously.

14. The method according to claim 13, wherein said effective amount of topotecan is administered three times within a 28-day period.

15. The method according to claim 13, wherein said rhenium chelate is administered 4 to 6 days after the administration of a first dose of said effective amount of topotecan.

16. The method according to claim 13, wherein said effective amount of topotecan is administered at a daily dose of from 1 mg/m² to 1.5 mg/m².

17. The method according to claim 1, wherein said effective amount of HDACi is administered orally before the administration of the said rhenium chelate.

18. The method according to claim 17, wherein said effective amount of HDACi is administered at a daily dose of from 75 mg/kg to 150 mg/kg.

19. The method according to claim 18, wherein said daily dose of HDACi is divided into 2 or 3 portions and is administered over 7 days.

20. The method according to claim 1, wherein said somatostatin subtype 2-expressing cancer is selected from a small cell lung carcinoma or a neuroendocrine carcinoma.
